(19) United States
(12) Patent Application Publication
(10) Pub. No.: US 2001/0051373 A1
(43) Pub. Date: Dec. 13, 2001

(54) T-CELL CLONES EXPRESSING ANTI-HIV-1 FACTORS

(76) Inventor: KUNAL SAHA, HILLIARD, OH (US)

Correspondence Address: THOMAS R ROLAND, VORYS SATER SEYMOUR & PEASE, 1828 L STREET NW, SUITE 111, WASHINGTON, DC, 20036

(21) Appl. No.: 09/369,333

(22) Filed: Aug. 6, 1999

(51) Int. Cl. C12N 5/08

(52) U.S. Cl. 435/372.3

(57) ABSTRACT

Certain clones of CD4+ cells from an HIV-infected patient who has not progressed to AIDS exhibit the ability to inhibit HIV replication at a stage subsequent to entry of the HIV-1 virus into the cell and integration of viral cDNA into the cellular genome. These cells secrete a soluble factor different from known cytokines and chemokines that can, inhibit infection of lymphocytes by SI HIV-1.

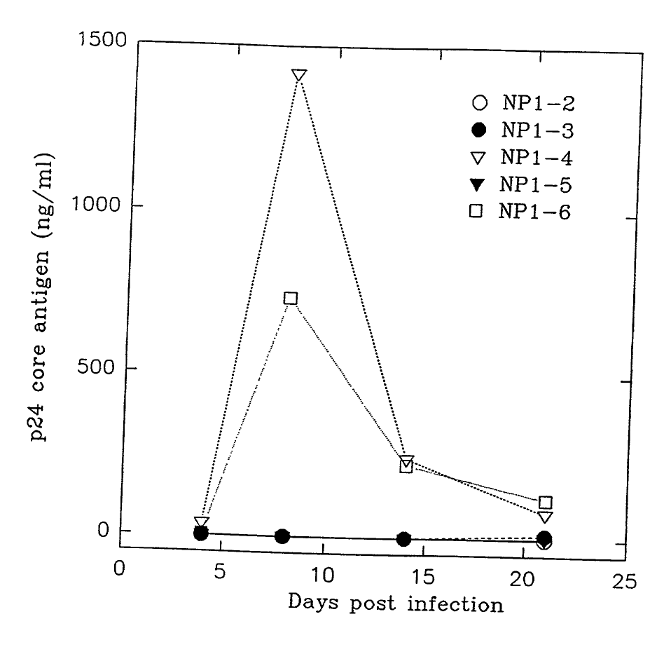

### BACKGROUND OF THE INVENTION

1. Field of the Invention

This invention relates to human cells that express factors capable of inhibiting infection of cells by HIV-1 virus and more particularly to human cell cultures that secrete cellular factors capable of inhibiting the infectivity of HIV-1 virus toward human and mammalian cells.

2. Brief Description of the Prior Art

Intense and widespread research is currently directed toward an understanding of the pathogenesis of Human Immunodeficiency Virus-1 (HIV-1) because of the importance of such knowledge in fighting the ongoing epidemic of HIV-1 infection and related Acquired Immune Deficiency Syndrome (AIDS).

The study of HIV pathogenesis has been stimulated by discoveries of chemokines and HIV-1 co-receptors. The co-receptor of macrophage-tropic or non-syncytium-inducing (NSI) strains of HIV-1, denominated as CCR5, was discovered in initial studies of two HIV-1-exposed but uninfected (EU) subjects whose CD4+ lymphocytes remained resistant to infection by NSI isolates of HIV-1. Further studies with these cells have shown that their resistance to infection by NSI viruses was due to high levels of RANTES, MIP-1α, and MIP-β, the ligands for CCR5, and a mutant CCR5 gene expression. Fusin, or CXCR4, the co-receptor for T-cell-tropic or syncytium inducing (SI) strains of HIV-1, was identified soon after the discovery of CCR5. SDF-1 was reported as a ligand for CXCR4 and was shown to be able to block infections with SI viruses in vitro. Studies on large cohorts have demonstrated that a homozygous deletion of the CCR5 gene is protective against HIV-disease progression primarily through resistance against NSI viruses. Conflicting reports of protection or enhancement of HIV-1 disease progression as a result of SDF-1 mutation have also been published. Although other chemokines/lymphokines such as MDC have also been shown to inhibit HIV-1 to some extent, the ligands for CCR5 and CXCR4 are the only factors known to have any real significance in vivo to prevent HIV-1 disease progression. However, several studies have indicated evidence of other as yet unknown cellular factors that may play important roles in suppressing HIV infections.

There are conflicting reports whether CD4+ cells can be differentially affected in HIV infection. Although some studies have suggested that selective depletion of T cells expressing TCR-Vβ sequences occurs in vitro, other reports have found no such selective depletion. Even though some evidence suggests that the course of HIV-1 infection can depend on genetic loci linked to major histocompatibility complex (HLA in humans) genes, no study thus far has shown whether different CD4+ cells from an individual patient can behave differently to HIV infection. The inventor and others have shown that endogenous production of β-chemokines by CD4+ cells from asymptomatic HIV-infected subjects can suppress HIV-1 replication. However, β-chemokines act only at the virus entry level by blocking binding of viruses to CCR5 and cannot inhibit virus replication once virus enters the cell. Furthermore, β-chemokines are not effective against SI viruses.

A major barrier to studies of functions of human T cells in HIV-infected patients has been the limited growth capacity of these cells in vitro. In recent years, Herpesvirus saimiri (HVS) has been used as a powerful tool to immortalize and study human T cells. The inventor has shown that CD4+ and CD8+ T cells from HIV-1 positive nonprogressors and AIDS patients can also be immortalized by HVS for long-term study.

However, continued progress in the understanding of HIV pathogenesis depends on the investigation, discovery, and isolation of factors that affect the infectivity of HIV-1 toward lymphocytes and the progression of HIV-1 infection in infected cells. Such studies can be greatly facilitated if suitable strains of cells are available that are adapted to express such factors under conditions wherein they can be conveniently studied.

Accordingly, a need has continued to exist for isolated cell lines that express factors affecting the pathogenesis of HIV-1 infection.

### SUMMARY OF THE INVENTION

A number of T-cell clones have now been developed from a single HIV-1 positive nonprogressor, that are phenotypically similar, but exhibit strikingly different behaviors when infected in vitro with SI viruses. Some of these clones are highly susceptible to SI viruses, while others are resistant to infection. In resistant CD4+ clones, the resistance to infection occurs at a stage after virus entry. Furthermore, the resistance is mediated, at least partially, by soluble factors produced by the resistant T cells that may not include the ligand for CXCR4, SDF-1 or other known cytokines.

Accordingly, it is an object of the invention to provide CD4+ cell clones useful in the study of HIV-1 pathogenesis.

A further object is to provide CD4+ cell clones that exhibit resistance to infection by SI HIV-1 viruses.

A further object is to provide CD4+ cell clones that exhibit resistance to infection by SI HIV-1 viruses at a stage after entry of the virus into the cell.

A further object is to provide CD4+ cell clones that produce soluble factors that mediate resistance of the cells to infection by SI HIV-1 viruses.

Other objects of the invention will become apparent from the description of the invention which follows.

### BRIEF DESCRIPTION OF THE DRAWING

The sole FIGURE illustrates the course of HIV infection of immortalized cell lines from a patient over time as indicated by the expression of p24 core antigen.

### DETAILED DESCRIPTION OF THE INVENTION AND PREFERRED EMBODIMENTS

The cell lines of the invention are clones derived from the PBLs of an HIV-positive nonprogressor. These cell lines do not produce HIV viruses and express cellular factors that inhibit the infectivity of HIV-1 virus toward human lymphocytes. As will be explained in the following discussion, the nature of these cellular factors is at present not known. However the effectiveness of these factors and their production by the cell lines of the invention are established by the experiments discussed below.

Generation of CD4+ T-cell clones:

The CD4+ T-cell clones of the invention were developed from a single nonprogressor patient (NP1) who has remained HIV-1 positive since 1983, having no symptoms and a stable CD4+ T-cell count of >1000 per microliter, without any anti-retroviral therapy. At the time of the generation of these clones, his viral load was not detectable.

The T-cell clones from the subject NP1 were developed by the procedures developed by Saha et al., and described in Immunol. Meth. 206:21-23 and Nat. Med. 2:1272-1275. Briefly, peripheral blood lymphocytes (PBLs) were isolated from heparinized blood by Ficoll-Hypaque gradient (Sigma Chemical, St. Louis, Mo.). Lymphocytes were separated by plastic adherent for 2 hours and resuspended in RPMI 1640 medium supplemented with 10% FCS, 2 mM L-glutamine, penicillin (100 U/ml), and streptomycin (100 μg/ml) (all from Life Technologies, Grand Island, N.Y.), and human IL-2 (20 units/ml) (Boehringer Mannheim, Indianapolis, Ind.). RO 31-8959, a HIV-1 protease inhibitor (generous gift of I. Duncan) was also added to the medium (final concentration of 10⁻⁵ mM) to inhibit HIV-1 spread during immortalization. Cells were immediately infected with HVS, group C, strain 488-77 (a gift from R. C. Desrosiers, Harvard Medical School) at a multiplicity of infection of 0.1. Three to five days after infection, HVS-infected cells were cloned by seeding at 0.5 to 1 cell/well containing 10⁵ X-irradiated allogeneic PBLs from normal donors in 96-well plates in a volume of 200 μl of the above medium. The protease inhibitor was maintained in the medium for the initial 3 weeks during the cloning process. The growing clones were expanded without any further stimulation with antigen/mitogen. MHCD4, an HVS-immortalized CD4+ clone from a normal donor and other CD4+ clones from an AIDS patient prepared by conventional techniques were also prepared.

Immunophenotyping of T-cell clones:

Immunophenotyping of the T-cell clones was performed on single cell suspensions by fluorescence-activated cell sorting (FACS), using a FACScan cytofluorograph (Becton Dickinson). The monoclonal antibodies (mAbs) used for phenotyping the T-cell clones included FITC- or PE-labeled OKT4 (anti-CD4), OKT3 (anti-CD3) anti-CD2, anti-CD14, anti-CD20, Tac (anti-CD25) (all from Biosource International, Camarillo, Calif.), BMA-031 (anti-TCR-αβ), and anti-CD69 (both from Becton Dickinson, San Jose, Calif.). Production of IFN-γ, IL-4, and HIV-1 p24 was determined by ELISA. The presence of HIV-1 DNA was determined by PCR. The phenotype of the CD4+ T-cell clones that were further investigated is listed in Table 1.

TABLE 1
Phenotype of CD4+ clones from NP1 patient

| Molecule | Expression |
|---|---|
| CD2 | + |
| CD3 | + |
| CD4 | + |
| CD8 | − |
| CD14 | − |
| CD20 | − |
| CD25 | + |
| CD69 | + |
| HLA-DR | + |
| TCR-α/β | + |
| IFN-α | − |
| IFN-γ | + |
| IL-4 | − |
| p24 (HIV-1) | − |
| HIV-1 DNA | − |

HIV-1 stocks and infection:

Both laboratory and primary isolates of SI viruses were used for infection of the CD4+ clones. The laboratory SI strain HTLV-IIIB (IIIB) (donated by R. C. Gallo, Univ. of Maryland) was propagated in H9 cells. Another laboratory SI strain, NL4/3 was also used and has been described by Chowdhury, I. H., et al., J. Virol. 70:5336-5345. Strain P13, a clinical isolate, has also been described previously by Sova, P., et al., J. Virol. 69:2557-2564 (1995). CD4+ clones were infected with SI viruses at 0.5 pg/cell for 2 hours at 37° C., washed two times and resuspended at 3×10⁵ cells/ml in 12-well plates. An HVS-immortalized CD4+ clone (MHCD4) from a normal donor, which is susceptible to IIIB virus infection was also used as a control. Viable cells were counted by trypan blue exclusion, and supernatants were collected at every 3-4 day intervals and stored at −80° C. HIV-1 production was measured by p24 ELISA using a commercial HIV Ag kit (Coulter, Hialeah, Fla.).

Cytokine assay:

Supernatants from four different CD4+ clones were tested for production of different cytokines including IL-4, -6, -10, -12, TNF-α, IFN-α, IFN-γ by ELISA using commercial kits. Supernatants from uninfected cells and from cells 4-7 days after infection (p.i.) were collected and assayed for cytokines.

RT-PCR detection of CXCR4 and SDF-1:

Cellular mRNA was prepared from equal numbers (5×10⁶) of cells from each clone using biotin-labeled oligo (dT)-coated magnetic beads from a mRNA isolation kit (Boehringer Mannheim, Indianapolis, Ind.) according to the manufacturer's instructions and treated with 10 U RNAase-free DNAse (Boehringer Mannheim). Random hexamer-primed cDNA was prepared from 50 82 g of mRNA of each sample using an RT-PCR kit (Perkin-Elmer Cetus, Norwalk, Conn.) and cDNA was amplified by 37 cycles of PCR (94° C. for 1 min, 60° C. for 1 min, and 72° C. for 2 min) followed by an additional cycle at 72° C. for 10 min for completion of polymerization in a volume of 50 μl using primers specific for CXCR4, SDF-1β, and β-actin (as internal control). The primer for SDF-1β was generated from human SDF-1 sequence (accession number U 16752) and primers for CXCR4 were generated by the procedure described by Liu, R., et al., Cell 86:367-377. The upstream and downstream primers (synthesized by Life Technologies) were as follows: CXCR4: 5′-GGCTAAAGCTTGGCCTGAGTGCTCCCAGTAGCC, 5′-CGTCCTCGAGCATCTGTGTTAGCTGGAGTG that gives a fragment of 1112 bp; SDF-β: 5′-CGCCAAGGTCGTGGTCGTGC, 5′-GCCCTTCAGATTGTAGCCCGGC that gives a fragment of 182 bp. The β-actin primers have been described previously in Saha, K., et al., Nat. Med. 2:1272-1275 (1996), and give a fragment of 838 bp. All samples were run with and without RT to rule out the possibility of any DNA contamination. MHCD4 and SupT1 cells were also used as controls. Amplified products were separated on 1.2% agarose gel in the presence of 0.5 μg/ml of ethidium bromide and photographed. In some experiments, mRNA was collected 2-3 days after infection with HIV to test whether SDF-1 may be induced after infection.

Fusion assays:

To test whether the CD4+ clones are functional and can efficiently fuse with HIV-1 envelope (‘env’) protein, fusion assays were performed by the procedure disclosed by Jonak, Z. L., et al., AIDS Res. Hum. Retro. 9:23-32. In this assay system, TF228.1.16, a human B lymphoid cell line which stably expresses functional HIV ‘env’ from IIIB virus is cocultured with CD4-expressing cells for 6-12 hours. Formation of syncytia in these cocultures indicates ‘env’-mediated membrane fusion and suggests that the CD4+ cells have functional receptors for SI viruses. The parental β lymphoid cell line BJAB, which does not express ‘env’ is used as a negative control in this assay.

Detection of HIV entry by PCR:

Whether the SI viruses can enter the resistant CD4+ clones was further tested by detection of HIV-specific sequences by PCR as described by Chowdhury, I. H., et al., J. Virol. 70:5336-5345. The CD4+ clones which were resistant to SI viruses were infected with DNAse-treated IIIB viruses and collected 12 hours after infection, washed and lysed for 1 hour at 60° C. in equal volumes of solution A (10 mM Tris [pH 8.3], 100 mM KCl, 2.5 mM MgCl₂) and solution B (10 mMTris [pH 8.3], 2.5 mM MgCl₂, 1% Tween 20, 1% NP-40, 60 μg of Proteinase K per ml) followed by incubation at 95° C. for 15 min. PCR was performed under standard conditions as described above using primer pairs SK38 and SK39 which amplify HIV ‘gag’ region, and BRUV3 and BRUV5 for ‘env’ region. The amplified products were run in a 1.2% agarose gel containing ethidium bromide (0.5 μg/ml) and photographed. For these experiments IIIB producing H9 cells and uninfected CD4+ clones were used for positive and negative controls respectively. In some experiments resistant clones were tested for the presence of HIV-1 DNA by PCR up to 5 weeks after infection.

Detection of HIV-suppression factors:

To test whether the HIV-resistant CD4+ clones were producing any diffusible anti-HIV factors, we made use of a transwell system as described previously by Saha, K., et al., J. Immunol. 157:3876-3885. In these experiments, cocultures were done in two-chambered wells separated by a 0.45 μm insert (Millipore, Bedford, Mass.) so that T cells in the upper and lower chambers do not come into direct contact with each other, but any soluble factor(s) produced by cells on either side may diffuse and act on cells on the other side. To rule out the possibility of any HVS-induced factors, HVS-transformed MHCD4 cells which are highly susceptible to infection with IIIB viruses and produce high levels of HIV-1 were used as the target. MHCD4 cells were infected with IIIB, washed and put (10⁵ cells/well) in the bottom chamber while an equal number (10⁵) of cells from different CD4+ clones from NP1 or only medium were put in the upper chamber. Supernatants were collected at various time points from the bottom chamber and assayed for p24 production as described above. The inhibition of HIV production by soluble factors expressed by five selected clones, NP1-2, NP1-3, NP1-4, NP1-5, and NP1-6, and their ability to inhibit HIV-1 are illustrated in Table 2.

TABLE 2
Inhibition of HIV Production by Soluble Factors from NP1 Clones

| Upper chamber | Lower chamber | p24 (ng/ml) |
|---|---|---|
| Medium only | MHCD4-NL4/3 cells | 114.7 |
| NP1-2 | ″ | 9.4 |
| NP1-3 | ″ | 11.9 |
| NP1-4 | ″ | 519 |
| NP1-5 | ″ | 79.4 |
| NP1-6 | ″ | 944 |

Results:

Characterization of CD4+ clones from NP1:

In general, the HVS-immortalized CD4+ T-cell clones that were derived from the nonprogressor patient, NP1, maintained an activated T-cell phenotype that was very similar to HVS-immortalized CD4+ clones from normal donors that have been described. All the CD4+ clones developed from NP1 had a surface phenotype consistent with activated (CD25+, CD69+, HLA-DR+), CD4+ T cells (CD4+, CD8−, CD14−, CD20−, CD2+, CD3+). Also, all of these clones expressed the T-cell receptor of αβ phenotype. PCR amplification using pairs of primers complementary to the 26 known TCR-Vβ sequences has revealed exclusive usage of specific Vβ in some of these and other CD4+ clones developed from HIV-infected subjects, indicating monoclonalities of these T-cell clones. However, no preference has been observed for any specific TCR-Vβ expansion in the T-cell clones either from normal donors or from HIV-infected subjects, suggesting that HVS does not act as a superantigen to immortalize T cells. Also like CD4+ clones from normal donors, all HVS immortalized CD4+ clones from NP1 expressed Th1 phenotype, i.e., producing IFN-γ but no IL-4.

As the CD4+ clones of this invention were developed from an HIV-1 infected patient, we tested these clones for the presence of HIV-1. None of the five CD4+ clones from NP1 selected produced any HIV-1 as determined by the presence of p24 core antigen or by coculture with HIV-permissive HeLa-CD4 or SupT1 cells. Also, none of these clones were positive for HIV-1 DNA as tested by PCR. However, immortalized CD4+ clones from HIV-1 positive patients that carry HIV-1 DNA without producing any virus have occasionally been observed. CD4 expression in various clones was also studied because the CD4 receptor, being the primary receptor for HIV-1, may play a critical role in HIV-1 infection. All five clones from NP1 expressed high and equivalent levels of CD4, which is comparable to the level of CD4 expression by HVS-immortalized clones from normal donors. Thus, all 5 CD4+ clones from patient NP1 were similar and they resembled CD4+ clones developed from normal donors.

Variable resistance against SI viruses:

The infectibility of the CD4+ clones from NP1 by SI was tested. As all of these clones were developed from a single patient expressing equivalent levels of CD4, it was expected that these clones would be equally infectible by SI viruses. Surprisingly, however, when a clinical SI isolate P13 was used for infection, a wide range of susceptibility among various NP1-clones was observed. While clones NP1-4 and NP1-6 were highly susceptible, producing peak p24 levels of over 1000 ng/ml, clones NP1-2 (ATCC . . . ), NP1-3 (ATCC . . . ), and NP1-5 (ATCC . . . ) were strongly resistant to infection with P13 viruses. The results of these infections are illustrated in the sole figure. The figure shows that clones NP1-4 and NP1-6 developed very high p24 levels at 8 days p.i., followed by the usual decline in p24 level as the infected cells died. On the contrary, clones NP1-2, NP1-3, and NP1-5 produced little or no p24 over the full 21 days of the experiment, indicating their strong resistance to infection by SI HIV-1. (Because the p24 levels of the resistant clone cultures are so low, the data points for these clones are almost coincident.) Similar results were obtained when laboratory isolate IIIB virus was used for infection. Clones NP1-4 and NP1-6 again produced high levels of virus, while clones NP1-3 and NP1-5 produced much lower levels of virus and almost no virus production was detected from clone NP1-2. Increased HIV production was accompanied by cell death in NP1-4 and NP1-6 clones. Overall these data demonstrate the clones NP1-4 and NP1-6 were highly infectible with SI viruses, while clones NP1-2, NP1-3, and NP1-5 were relatively or completely resistant to infection with SI viruses.

HIV-resistance is not at virus entry level:

Resistance against NSI viruses in CD4+ cells from EU subjects has been found at virus entry level due to mutant CCR5 co-receptors expression. It was considered to be possible that a similar mechanism might be involved in resistance against SI viruses in the NP1 clones, although it was apparent that a global defect in the co-receptor was very unlikely in this case because some of the clones from the same donor were susceptible to infection. Accordingly, expression of mRNA of the co-receptor CXCR4 in the resistant as well as susceptible clones from NP1 was investigated. No significant difference in the levels of CXCR4 mRNA expression was observed among different susceptible (e.g., NP1-6) or resistant (e.g., NP1-3) clones from NP1. HVS-immortalized and SI-susceptible CD4+ clones from normal donors (MHCD4) or from AIDS patients (AD1-13 and AD1-22) also expressed comparable mRNA levels of CXCR4 co-receptor. Indeed, using anti-CRCX4 antibody, no difference was found in the expression of CXCR4 in several resistant or susceptible CD4+ clones by FACS analysis. These results demonstrate that co-receptor CXCR4 may not be involved in the difference in infection observed in the various NP1 clones. Further tests were conducted to determine whether the resistant and susceptible clones from NP1 produced different levels of SDF-1. Both resistant (NP1-6) and susceptible (NP1-3) clones expressed equivalent levels of SDF-1, as did the clone MHCD4 from a normal donor and SI virus susceptible clones AD1-13 and AD1-22 from an AIDS patient. Indeed, no significant change in SDF-1 production was observed in any of these or other clones from NP1 even after infection with SI viruses. Thus, the resistance against SI viruses in the clones from NP1 may not be mediated at the virus entry level either through inadequate expression of the co-receptor CRCX4 or by overproduction of SDF-1.

Further evidence that inhibition of entry of SI viruses in the resistant clones from NP1 may not involve surface receptors comes from the “fusion” assays. In this system, ‘env’-expressing TF228.1.16 cells were cocultured with different NP1 clones to test the efficiency of ‘env’-mediated fusion. In a representative experiment with one of the resistant clones NP1-2, efficient fusion took place when these resistant T cells were cocultured with TF228.1.16 cells, as was evident by the formation of prominent syncytia, whereas no syncytia formed when the same clone was cocultured with control cells, BJAB that did not express ‘env’. All other NP1 clones, whether resistant or susceptible to SI viruses, or clones from normal donors were also able to fuse efficiently when cocultured with TF228.1.167 cells, indicating that the resistant clones were competent for the entry of the viruses.

Finally, the resistant clones were also examined for the presence of HIV-1 DNA after non-productive infection with SI viruses. Using HIV-specific primers and PCR it was discovered that although the resistant clone, e.g., NP1-2, did not produce any virus, these cells became positive for ‘gag’ and ‘env’ after infection with IIIB viruses, indicating that these viruses could enter the cells, be reverse transcribed and integrated. Indeed, the resistant clones were found to be positive for HIV-1 DNA as long as 5 weeks after infection with SI viruses. Taken together these results suggest that resistance against SI viruses observed in NP1-2, NP1-3 or NP1-5 clones involves a mechanism that acts after virus entry, reverse transcription and integration.

HIV-suppression factors:

CD4+ cells from asymptomatic HIV-infected subjects can protect themselves against NSI viruses by overproduction of β-chemokines. As discussed above, overexpression of SDF-1, the ligand for CXCR4, was not observed in any of the resistant clones. The above results also show that resistance against SI viruses probably occurs at a level after virus entry and integration. Therefore, factors like SDF-1 that can only inhibit HIV by blocking the co-receptors may not be involved in the mechanism of resistance in these clones. In order to test whether inhibition of SI viruses in the resistant clones might be mediated by other soluble factors, an NL4/3-infected MHDC4 clone was used, as these HVS-immortalized cells have been shown to be highly productive for SI viruses. In two-chambered experiments, NL4/3-infected MHCD4 cells were separated by a semi-permeable membrane from different resistant or susceptible clones from NP1. It was observed that HIV production by MHCD4 cells in the bottom chamber was strongly inhibited when either of the resistant clones NP1-2 or NP1-3 was placed in the upper chamber. Virus production was also inhibited, albeit at a lower level, when the other resistant clone NP1-5 was put in the upper chamber. In contrast, when either of the two susceptible clones NP1-4 or NP1-6 was put in the upper chamber, no inhibition of virus production was observed. Indeed, the level of HIV-1 production was enhanced when clone NP1-4 or NP1-6 was put in the upper chamber, probably as a result of reinfection of these clones. Taken together, these results suggest that soluble factors produced by resistant NP1 clones have potent suppression activities against SI viruses that can work at a stage after virus integration. However, whether these anti-viral factors are produced constitutively or only after stimulation with HIV-1 infection remains unclear, as when supernatants from the resistant clones were directly added to the NL4/3-infected cells, no significant inhibition of virus production was observed, indicating that the suppression factors may be produced only after triggering with HIV antigens. However, it is also possible that the HIV suppression factors that are produced constitutively by the resistant clones have a short half-life and need continuous supply for effective HIV inhibition.

Cytokines such as IFN-α have been shown to inhibit HIV replication. Accordingly, a panel of cytokines was tested that may influence replication of SI viruses. No significant differences in the levels of expression of different cytokines among the resistant (NP1-2, -3, -5) or susceptible (NP1-4, -6) were observed whether before or after infection with SI viruses. The results of these tests are summarized in Table 3, wherein the R/S column indicates whether the clone was resistant (R) or susceptible (S) to infection, and the values give the cytokine production levels before infection (without parentheses) and after infection (in parentheses) by primary SI HIV-1 virus strain P13.

TABLE 3
Cytokine Production (pg/ml) by CD4+
Clones Before and After
Infection with Primary SI HIV-1 Virus Strain P13

| | | | | | | | IFN- | |
|---|---|---|---|---|---|---|---|---|
| Clone | R/S | IL-4 | IL-6 | IL-10 | IL-12 | TNF-α | α | IFN-γ |
| NP1-2 | R | 0 | 358 | 0 | 0 | 982 | 0 | 4283 |
| | | (0) | (145) | (0) | (66) | (316) | (0) | (1759) |
| NP1-3 | R | 0 | 3326 | 0 | 107 | 535 | 0 | 2089 |
| | | (0) | (5911) | (0) | (85) | (1850) | (0) | (4593) |
| NP1-4 | S | 0 | 547 | 0 | 60 | 2394 | 0 | 11881 |
| | | (0) | (625) | (28) | (47) | (1787) | (0) | (11609) |
| NP1-5 | R | 0 | 839 | 73 | 0 | 683 | 0 | 8667 |
| | | (0) | (1048) | (51) | (0) | (756) | (0) | (12005) |
| NP1-6 | S | 0 | 485 | 53 | 0 | 1503 | 0 | 7650 |
| | | (0) | (438) | (56) | (140) | (805) | (0) | (3596) |

All of the clones, whether resistant or susceptible, expressed no IL-4, and IFN-α and little or no IL-10. Resistant clone NP1-3 expressed high levels of IL-6 that increased even further after infection. However, other resistant clones NP1-2 and NP1-5 produced amounts of IL-6 that were comparable to that of susceptible clones NP1-4 and NP1-6, suggesting that IL-6 may not be involved in the resistance of these clones. All of the clones also constitutively expressed moderate to high levels of TNF-α and IFN-γ that did not change significantly after infection. However, no definite relationship was observed between production of any of these cytokines and resistance or susceptibility to SI viruses. Thus, it appears that none of these cytokines is involved in the resistance against SI viruses in the selected CD4+ clones.

Discussion:

CD4+ lymphocytes can protect themselves against NSI viruses by virtue of increased β-chemokine production or the expression of mutant CCR5 co-receptor. No such resistance in CD4+ cells against SI viruses has been published hitherto. By studying different CD4+ clones from an HIV-1 positive nonprogressor it can be learned that selected CD4+ cells can be resistant against SI viruses. It has been found that the resistance against SI viruses in thee CD4+ cells is not due to defective co-receptor (CXCR4) expression or overproduction of SDF-1. Finally, evidence suggests a novel mechanism of resistance against SI viruses in these clones that acts at a stage after virus entry and mediated, at least partially, by soluble factors.

CD4+ clones from NP1, an HIV-1-positive nonprogressor can be selectively resistant or susceptible to infection with SI viruses. While clones NP1-4 and NP1-6 were highly susceptible, clones NP1-2 (ATCC . . . ), NP1-3 (ATCC . . . ), and NP1-5 (ATCC . . . ) were relatively or completely resistant to SI viruses. Although previous studies have reported that HIV can selectively deplete CD4+ T cells expressing specific TCR-Vβ sequences, the reason for this selective depletion was not due to enhanced HIV replicative ability, but rather due to possible superantigen like activity of HIV antigens causing apoptosis of selected CD4+ cells. Establishment of HIV-resistant and -susceptible clones from a single nonprogressor indicates that CD4+ cells can behave differently to HIV infection in vivo. It is not likely that an aberrant molecule induced by HVS-immortalization of NP1 clones was responsible for such resistance against HIV for several reasons. First, HVS has been used as a powerful tool in the studies of T-cell functions over the past several years and HVS-immortalized T cells behave much like primary T lymphocytes. By and large, HVS-immortalized T-cells remain phenotypically unchanged and maintain their normal functional activities. Thus there is little evidence of aberrant gene expression in T cells immortalized by HVS. Second, only two viral proteins have been detected from HVS-immortalized T cells thus far, with little or no homologies with any known HIV-suppression factors. Third, it has been shown that HVS-immortalized T cells are fully permissive for replication of HIV, even for some strains with restricted host range, indicating that there may be no inherent incompatibility between HVS-immortalization and HIV infection. Finally, as clones immortalized by HVS from the same donor were found to be either resistant or susceptible to SI viruses, factors induced by HVS were unlikely to play any role in the observed resistance.

A mutant CCR5 allele has been found responsible for resistance against NSI viruses in large cohort studies. Although no such mutation of CXCR4 gene has been described so far, it was found that all NP1 clones, whether resistant or susceptible to SI viruses expressed comparable levels of CXCR4 mRNA. All NP1 clones also expressed a normal CCR5 gene. Other studies have suggested that CXCR4, CCR5, and related CCR3, CCR2B are probably not the only co-receptors for HIV, and other as yet unidentified co-factors may also be involved in the pathogenesis of HIV. It is unlikely that a global defect (mutation) in co-receptors expression may be involved in the resistance of NP1 clones as both the resistant and the susceptible were developed from a single individual. Further evidence that the resistance against SI viruses was not due to a block at the level of virus entry comes from the “fusion” assays. Both the resistant and susceptible clones were able to form syncytia efficiently with cells expressing ‘env’ from IIIB viruses, indicating that the resistant clones did not have any defect towards mediating virus-cell fusion. Additionally, detection of HIV-1 DNA in the resistant clones after non-productive infection also suggests that these viruses could enter the cells efficiently and the block in virus production probably occurred after virus entry and integration. Interestingly, the levels of CD4 expression remained high in the resistant clones even after they became HIV DNA-positive. Finally, resistant clones like NP1-3 and NP1-5 produced low but detectable levels of virus after infection with some isolates of the SI viruses, indicating that these clones were not completely but relatively resistant against these viruses, which may not be caused by a defect in the virus entry. Taken together, these results strongly suggest that, unlike the previously described CCR5 mutant clones from EU subjects, the resistance in clones from NP1 involves a mechanism that acts at a post-entry level.

Kinter et al., Proc. Natl. Acad. Sci. USA 93:14076-14071 (1996) have shown that bulk CD4+ cells from HIV-infected asymptomatic subjects were able to inhibit HIV replication by producing increased levels of β-chemokines. Indeed, it has recently been reported (Saha, K., et al., J. Virology 72:876-881 (1998)) that all NP1 clones, whether resistant or susceptible to SI viruses, also produced high levels of β-chemokines as well, and were for the most part resistant against infection with NSI viruses. However, β-chemokines can act only against NSI viruses. Also, as discussed above, the mechanism of resistance in NP1 clones against SI viruses probably acted at a stage after virus entry, and thus may not involve increased ligand (SDF-1) production. However, it might be argued that overproduction of SDF-1 could still inhibit reinfection, and thus can suppress eventual virus production. Testing revealed no difference in the levels of CXCR4 or SDF-1 expression in the resistant or susceptible clones whether before or after infection with SI viruses. Indeed, CXCR4 and SDF-1 expression in other HVS-transformed and susceptible clones was comparable to that of the NP1 clones, indicating that resistance in NP1 clones may not involve decreased expression of CXCR4 co-receptor or increased production of SDF-1. Furthermore, as demonstrated by the transwell experiments, the resistant and not the susceptible clones produced soluble factors that could inhibit production of SI viruses from chronically infected cells, indicating that the anti-viral effects of the factors produced by the resistant clones can act at a stage after virus integration. Cytokines such as IFN-α have been shown to downregulate HIV replication after de novo infection at an early stage prior to integration. IL-10 can also inhibit HIV replication from macrophages by inhibiting autocrine production of TNF-α and IL-6 but has no anti-viral effects on T-cells. However, none of these cytokines is known to inhibit HIV replication as strongly as the factors from resistant NP1 clones, e.g., NP1-2, NP1-3, did. Additionally, by ELISA no difference was observed in the expression of different cytokines (IL-2, IL-4, IL-6, IL-10, IL-12, TNF-α, IFN-γ) between resistant and susceptible clones whether before or after infection with SI viruses. Thus, the nature of the factor(s) produced by resistant NP1 clones that inhibit SI viruses remain unknown at the present time. It has been shown that CD8+ cells from HIV-1 infected subjects produce factors that can inhibit virus replication (Kinter et al., Proc. Natl. Acad. Sci. USA 93:14076-14071 (1996); Mackewicz, C. E., et al., Science 274:1393-1394 (1996); Moriuchi, H., et al., Proc. Natl. Acad. Sci. USA 93:15341-15345 (1996)). Using HVS-immortalized cells from an HIV-infected asymptomatic individual, it has also been shown that soluble factors other than β-chemokines produced by CD8+ cells can suppress HIV (Moriuchi, H., et al., Proc. Natl. Acad. Sci. USA 93:15341-15345 (1996)). The inventor has now shown that soluble factors that may not include known cytokines/chemokines and are produced by the CD4+ cells from an HIV-infected nonprogressor can inhibit SI viruses at a stage after virus entry. The invention having now been fully described, it should be understood that it may be embodied in other specific forms or variations without departing from its spirit or essential characteristics. Accordingly, the embodiments described above are to be considered in all respects as illustrative and not restrictive, the scope of the invention being indicated by the appended claims rather than by the foregoing description, and all changes which come within the meaning and range of equivalency of the claims are intended to be embraced therein.

I claim:

1. A CD4+ T-cell clone capable of producing soluble factors other than IFN-α that can inhibit replication of SI HIV-1 in infected T cells.

2. The CD4+ T-cell clone of claim 1 wherein said T-cell clone is derived from an HIV-1 infected nonprogressor.

3. The T-cell clone of claim 1 wherein said T-cell clone is NP1-2 (ATCC . . . ).

4. The T-cell clone of claim 1 wherein said T-cell clone is NP1-3 (ATCC . . . ).

5. The T-cell clone of claim 1 wherein said T-cell clone is NP1-5 (ATCC . . . ).
